(19) United States
(12) Patent Application Publication
(10) Pub. No.: US 2026/0283509 A1
(43) Pub. Date: Sep. 24, 2026

(54) EXTRUSION-PRINTED BIOCHEMICAL SENSOR, WEARABLE DEVICE, AND SYSTEM FOR DETERMINING REPRODUCTIVE STATUS OF USER

(71) Applicant: FIBRA INC., Vaughan (CA)

(72) Inventor: Parnian MAJD, Vaughan (CA)

(21) Appl. No.: 19/476,009

(22) PCT Filed: Apr. 9, 2024
(86) PCT No.: PCT/IB2024/053472

§ 371 Date: Oct. 16, 2025

Related U.S. Application Data
(60) Provisional application No. 63/460,361, filed on Apr. 19, 2023.
(60) Provisional application No. 63/529,399, filed on Jul. 28, 2023.

(51) Int. Cl. A61B 5/145; A61B 5/00; A61B 5/01; A61B 5/024; A61B 5/08; A61B 5/1468

(52) U.S. Cl. CPC A61B 5/14507; A61B 5/01; A61B 5/024; A61B 5/0816; A61B 5/14539; A61B 5/14546; A61B 5/1468; A61B 5/6804; A61B 2562/0271; A61B 2562/125; A61B 2562/164

(57) ABSTRACT

An extrusion-printed biochemical sensor for detecting properties of a biological liquid is disclosed. The sensor includes a plurality of electrodes (including a first and second electrode) formed from a conductive material that has been extrusion-printed onto a flexible substrate. In response to a biological liquid contacting the electrodes, the electrodes are configured to transmit a feedback signal to a microcontroller which determines a biochemical property of the biological liquid based on the feedback signal. The biochemical sensor may be integrated into a wearable device for measuring biochemical properties of liquids secreted by the user.

### FIELD

The present specification is directed to wearable devices, and in particular, extrusion-printed biochemical sensors for wearable devices.

### BACKGROUND

Wearable devices or smart clothes primarily involve the integration of screen-printed sensors directly onto fabric substrates, enabling the real-time monitoring of various physiological parameters such as heart rate, body temperature, and muscle movement, without compromising the comfort or wearability of the garment. While these devices, known for their flexibility and conformity to different body shapes, have made strides in areas like fitness tracking and medical monitoring, the development of practical biochemical sensors for vital health metrics integration remains a challenge yet to be overcome.

### SUMMARY

According to one aspect, the present specification provides an extrusion printed biochemical sensor. The biochemical sensor includes a flexible substrate, a plurality of electrodes, and a microcontroller. The electrodes include a conductive material which is extrusion-printed onto the flexible substrate. The plurality of electrodes include a first electrode and a second electrode spaced from the first electrode. The microcontroller is connected to the plurality of electrodes and is configured to receive a feedback signal from the second electrode responsive to a biochemical property of a biological liquid that contacts the first electrode and the second electrode. Based on the feedback signal, the microcontroller is configured to determine the biochemical property of the biological liquid.

In some examples, the conductive material includes at least one of silver, gold, copper, carbon nanotubules (CNT), reduced graphene oxide (rGO), polyaniline (PANI), poly(3,4-ethylenedioxythiophene), and polystyrene sulfonate (PEDOT:PSS).

In other examples, the flexible substrate includes a fabric blend including polyester and cotton.

In further examples, the biochemical property could be the acidity of the biological liquid, and the microcontroller further includes a potentiostat configured to measure the potential across the first electrode and the second electrode to determine the acidity of the biological liquid.

In yet further examples, the biochemical property is the volume of the biological liquid, and the microcontroller is configured to apply a test signal to the first electrode and receive the feedback signal from the second electrode, the feedback signal responsive to the test signal. The microcontroller further retrieves from memory a gap distance between the first and second electrode and determines the volume of the biological liquid based on a gap distance between the first electrode and the second electrode.

In some examples, the biochemical sensor further comprising a plurality of the second electrodes, and the microcontroller is configured to determine the volume of the biological liquid based on the number of second electrodes transmitting the feedback signal.

In other examples, the biochemical property is the salt concentration of the biological liquid, and the microcontroller is further configured to apply a test signal to the first electrode and receive the feedback signal from the second electrode, the feedback signal responsive to the test signal. Based on the test signal and the response signal, the microcontroller is configured to measure the conductivity of the biological liquid using a conductivity meter. Based on the conductivity, the microcontroller is configured to determine the salt concentration of the biological liquid.

According to another aspect, the present specification provides a wearable device which includes a garment including a flexible substrate and configured to be worn by a user. The wearable device further includes a biochemical sensor including a plurality of electrodes. The plurality of electrodes includes a conductive material extrusion-printed onto the flexible substrate. The plurality of electrodes includes a first electrode and a second electrode spaced from the first electrode. The wearable device further includes a microcontroller connected to the plurality of electrodes and configured to receive a feedback signal from the second electrode responsive to a biochemical property of a biological liquid which contacts at least the first electrode and the second electrode. The microcontroller is further configured to determine the biochemical property of the biological liquid based on the feedback signal.

In some examples, the conductive material includes at least one of silver, gold, copper, carbon nanotubules (CNT), reduced graphene oxide (rGO), polyaniline (PANI), and poly(3,4-ethylenedioxythiophene) polystyrene sulfonate (PEDOT: PSS).

In other examples, the flexible substrate includes a fabric blend including polyester and cotton.

In other examples, the biochemical property could be the acidity of the biological liquid, and the microcontroller further includes a potentiostat configured to measure the potential across the first electrode and the second electrode to determine the acidity of the biological liquid.

In other examples, the biochemical property is the volume of the biological liquid, and the microcontroller is configured to apply a test signal to the first electrode and receive the feedback signal from the second electrode, the feedback signal responsive to the test signal. The microcontroller is further configured to retrieve a gap distance between the first electrode and the second electrode from memory and determine the volume of the biological liquid based on the gap distance.

In other examples, the wearable device further includes a plurality of the second electrodes, and the microcontroller is further configured to determine the volume of the biological liquid based on the number of second electrodes transmitting the feedback signal.

In further examples, the biochemical property is the salt concentration of the biological liquid, and the microcontroller further includes a conductivity meter configured to measure the conductivity of the biological liquid between the first electrode and the second electrode and determine the salt concentration of the biological liquid based on the conductivity.

In yet further examples, the wearable device further includes a wireless transmitter connected to the microcontroller, the wireless transmitter configured to transmit the feedback signal via a network.

In other examples, the wearable device further includes a temperature sensor positioned to contact the user's skin. The temperature sensor includes a pair of electrodes connected to the microcontroller and a temperature-sensitive compound extrusion-printed onto the flexible substrate and positioned to connect with the pair of electrodes. The microcontroller controller is configured to measure an electrical property of the temperature-sensitive material and determine the skin temperature of the user based on the electrical property.

In further examples, the temperature-sensitive compound includes poly(3,4-ethylenedioxythiophene) polystyrene sulfonate (PEDOT: PSS) and graphene oxide (GO), and the pair of electrodes include silver.

In yet further examples, the wearable device includes a piezoelectric sensor connected to the microcontroller and configured to transmit piezoelectric signals to the microcontroller responsive to the user's heart rate or breath rate.

According to another aspect, the present specification provides a method of manufacturing a biochemical sensor using an extrusion printer. The method includes heating a conductive material with a heating element of the extrusion printer, extruding the conductive material through a nozzle of the extrusion printer to form a first electrode and a second electrode on a flexible substrate, and connecting the first electrode and the second electrode to respective connectors.

In some examples, the flexible substrate includes a fabric blend of polyester and cotton.

In other examples, the conductive material includes a silver ink.

In further examples, the heating temperature is about 120° C. and the heating time is about 30 minutes.

In yet further examples, the method includes stabilizing the flexible substrate with a vacuum while extruding the conductive material.

In other examples, the method includes controlling the pressure in the nozzle while extruding the conductive material.

According to another aspect, the present specification provides a system for managing feedback data representing physiological parameters of reproductive cycles. The system includes a plurality of wearable devices including a first wearable device, the first wearable device including an input device configured to measure physiological parameters. The system also includes a fertility tracking engine configured to receive the feedback data from the wearable device, retrieve reference data from memory, compare the feedback data to the reference data, and determine the reproductive status of the user based on the comparison.

In other examples, the physiological parameters include one or more of: acidity of a biological liquid, skin temperature, breath rate, pulse rate, salt content of a biological liquid, lactic acid content of the biological liquid, antibody content of the biological liquid, viscoelasticity of the biological liquid, and volume of the biological liquid.

In other examples, the system includes a network for transmitting the feedback data from the wearable device to the fertility tracking engine.

In other examples, the system includes a computing device associated with the user and connected to the network, wherein the computing device is configured to receive the reproductive status from the fertility tracking engine and display the reproductive status.

In other examples, the feedback data further represents at least one of: an identifier associated with the user, the date on which a feedback signal was received at a wearable device, and user-reported data.

In examples where the feedback data represents the user-reported data, the user-reported data may include at least one of: dietary sugar, age, ethnicity, age of first menstruation, method of birth control, reproductive and hormonal disorders, mood, pain, sleep quality, sleep duration, sex drive, sexual activity, menstrual bleeding, spotting, vaginal discharge, skin condition, cravings, digestion, weight, and hair condition.

In other examples, the fertility tracking engine is further configured to forecast the reproductive cycle of the user based on the comparison of the feedback data to the reference data. The forecast includes a prediction for one or more future reproductive statuses.

In other examples, comparing the feedback data to the reference data includes processing the feedback data using an algorithm trained to identify patterns in the reference data.

In other examples, the algorithm includes a machine learning algorithm, a deep-learning-based algorithm, or a neural network.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Embodiments are described with reference to the following figures.

FIG. 1 is a perspective view of a biochemical sensor, according to one embodiment.

FIG. 2 is a perspective view of another biochemical sensor according to another embodiment.

FIG. 3 is a perspective view of a further biochemical sensor according to a further embodiment.

FIG. 4 is a block diagram of a method of manufacturing the biochemical sensor of FIG. 1.

FIG. 5 is a front elevation view of a wearable device including the biochemical sensor of FIG. 1.

FIG. 6 is a block diagram of the wearable device of FIG. 5.

FIG. 7 is a schematic diagram of a network including the wearable device of FIG. 5 and a server.

FIG. 8 is a schematic diagram of the server of FIG. 6.

FIG. 9 is a flowchart of a method of determining a reproductive status using the system of FIG. 7.

FIG. 10 is a flowchart of a method of training a machine learning algorithm using the system of FIG. 7.

FIG. 11 is a photograph of a water droplet deposited onto a cotton-polyester fabric.

FIG. 12 is a graph showing the resistance in a temperature sensor, according to one embodiment.

### DETAILED DESCRIPTION

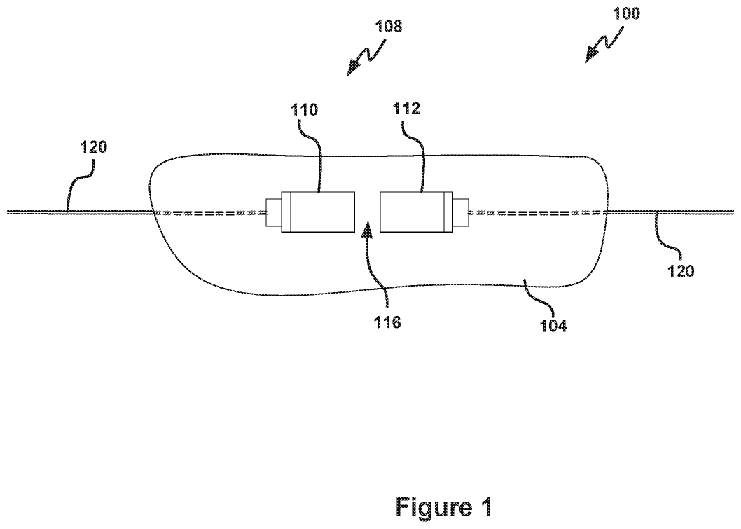

FIG. 1 is a schematic diagram of a biochemical sensor 100 according to one embodiment. As will be described herein, the biochemical sensor 100 is configured to characterize a biological liquid by measuring a biochemical property thereof.

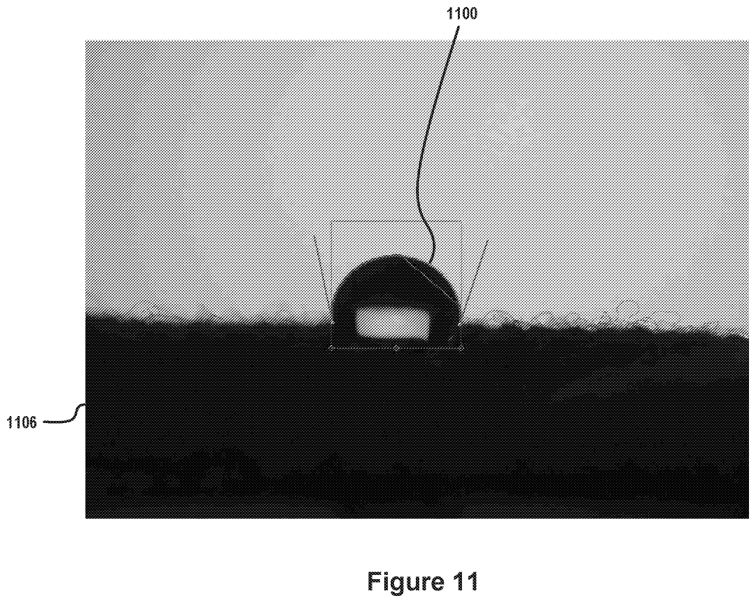

The biochemical sensor 100 is printed onto a flexible substrate 104. The flexible substrate may include woven fabrics and non-woven fabrics. Specific, non-limiting examples of substrates include polyamide film, polyethylene naphthalate (PEN) film, leather, latex, cotton, silk, linen, polyester, ethylene polyester (PET), nylon, rayon, modal, and combinations thereof. In some embodiments, the flexible substrate 104 comprises a hydrophobic material. The hydrophobic material may include nylon, polyester, polyamide film, the like or combinations thereof. A specific example of a polyamide film is Kapton™ (Dupont). In specific non-limiting examples, the flexible substrate comprises a fabric blend of cotton and polyester, and in particular examples about 10% polyester and about 90% cotton. As shown in FIG. 11, when water is deposited on a fabric comprising 10% polyester and 90% cotton, the average contact angle between the water droplet 1100 and the blended fabric 1104 is 105.15°. In other embodiments, the flexible substrate 104 is selected to optimize the distribution and drying time of the extrusion printed material. The drying time for cotton is generally faster than the drying time for nylon. The distribution of extrusion printed materials is generally better on nylon than cotton. As will be described in further detail herein, the flexible substrate 104 may comprise a garment configured to be worn by a user. In other examples, the flexible substrate 104 comprises an insert or liner configured to be worn in a garment.

The biochemical sensor 100 comprises a plurality of electrodes 108 (referred to generally as “electrode 108” and collectively as “electrodes 108”) formed on the surface of the flexible substrate 104. The plurality of electrodes 108 include a first electrode 110 and a second electrode 112 spaced from the first electrode 110. The distance between the first electrode 110 and the second electrode 112 will be referred to herein as the gap distance 116. As will be explained with respect to the examples provided herein, the number of electrodes is not limited to two, and the biochemical sensor 100 may include any suitable number of the electrodes 108. The biochemical sensor 100 is configured to characterize a biological liquid that is deposited onto or absorbed by the flexible substrate 104 such that the electrodes 108 are electrically connected by the biological liquid. The biological liquid is not particularly limited and may include sebum, sweat, vaginal discharge, cervical mucus, urine, blood, amniotic fluid, lochia, or the like.

The electrodes 108 comprise a conductive compound which has been extrusion-printed onto the flexible substrate 104. Extrusion printing is suitable for layering printed materials, and the electrodes 108 may comprise one or more layers printed onto the flexible substrate 104. The conductive compound may comprise a conductive metal such as copper, gold, silver, or the like. The conductive compound may comprise an organic material including but not limited to carbon nanotubules (CNT), reduced graphene oxide (rGO), polyaniline (PANI), poly(3,4-ethylenedioxythiophene) polystyrene sulfonate (PEDOT:PSS), and combinations thereof. PANI is easy to synthesize, cost-effective, and may improve sensitivity to pH changes. Carbon nanotubes can demonstrate high electrical conductivity and mechanical strength, which can improve the sensor's stability. Graphene oxide has a high surface area and good electrical conductivity, which can contribute to enhanced sensitivity in the biochemical sensor 100. PEDOT: PSS demonstrates good conductivity, transparency, and flexibility, making it suitable for flexible sensors. The conductive compound may further comprise at least one binder, surfactant, ligand, or solvent. In particular examples, the conductive compound comprises a viscous silver ink. The viscous silver ink may have a viscosity of about 85000 cps. The viscous silver ink may contain about 72% silver after curing, less than about 10 ppm chloride, less than about 10 ppm sodium, and less than about 10 ppm potassium. Suitable silver inks may be sourced from Creative Materials™ or NovaCentrix™, however others are known in the art.

The electrode 108 may further include a cover layer applied over the conductive material. The cover layer may be applied using extrusion printing, adhesives, sewing, or other techniques known in the art. Non-limiting examples of the cover layer include cotton, polyvinyl alcohol (PVA) hydrogel, polyvinyl alcohol/polyacrylic acid (PAA/PVA) hydrogel, filter paper, and combinations thereof.

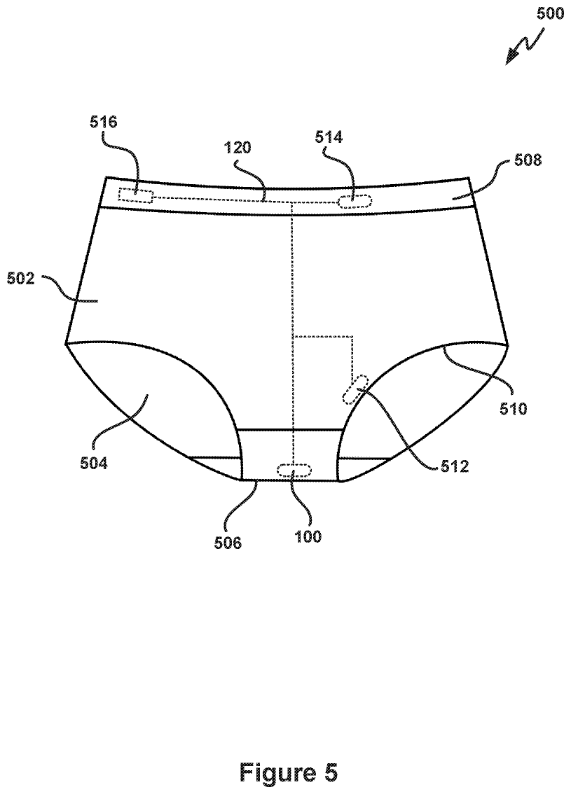

The electrodes 108 are connected to a microcontroller, which will be explained in greater detail herein with respect to FIG. 5. The microcontroller is configured to receive a feedback signal from the second electrode responsive to the biochemical property of the biological liquid contacting at least the first electrode 110 and the second electrode 112. Based on the feedback signal, the microcontroller is further configured to determine the biochemical property of the biological liquid.

The electrodes 108 may be connected to the microcontroller with connectors 120 (referred to generally as “connector 120” or collectively as “connectors 120”). Each of the electrodes 108 is electrically connected to at least one connector 120. In the embodiment shown in FIG. 1, the first electrode 110 and the second electrode 112 are each connected to three connectors 120. The connectors 120 may comprise any suitable conductive material such as copper, silver, gold or conductive yarn. In some examples, the connectors 120 comprise copper, silver, or gold wires. The gauge or the wire may be selected to retain flexibility in the biochemical sensor 100. In specific examples, the gauge of the wire is 34 AWG. In other examples, the connectors 120 comprise a conductive yarn comprising silver, stainless steel, or another suitable material. In specific non-limiting examples, the conductive yarns have a 0.5 mm diameter. A coating may cover the connector 120 to protect the connector from oxidization.

The biochemical sensor 100 may be selected to be small, washable, flexible, and sufficiently durable to withstand deformation of the flexible substrate 104.

The conductive compound and gap distance 116 may be selected according to the biochemical property that is measured by the biochemical sensor 100. The biochemical property may include but is not limited to acidity, volume, salt concentration, antibody concentration, lactic acid concentration, viscoelasticity, and combinations thereof.

In specific, non-limiting examples, the biochemical sensor 100 is configured to measure the acidity of the biological liquid. In these examples, the first electrode 110 is a reference electrode and the second electrode 112 is a working electrode. The microcontroller includes a potentiostat for measuring the electrical potential across the reference electrode and the working electrode, the potential indicating the pH. Based on the feedback signal received from the second electrode, the microcontroller is configured to measure the potential of the biological liquid. The microcontroller is further configured to determine the acidity of the biological liquid based on the potential.

In examples where the biochemical sensor 100 is configured to measure acidity (pH), the conductive compound may comprise polyaniline (PANI), poly(3,4-ethylenedioxythiophene), polystyrene sulfonate (PEDOT:PSS), the like, and combinations thereof. In specific non-limiting examples, the working electrode is coated with a mixture of PANI and PEDOT: PSS. Other conductive materials like silver, carbon nanotubule (CNT), and the like may be incorporated into the acidity sensor to enhance the electron transfer and signal output.

In examples where the electrodes 108 comprise PEDOT: PSS, the conductive compound may comprise PEDOT: PSS mixed with water to form a suspension which is extrusion-printed onto the flexible substrate 104.

The overall impedance of the biochemical sensor 100 will change when the flexible substrate 104 absorbs different amounts of liquid. With a single-layer electrode, the sensing range is from about 15 μl to about 75 μl, and the sensitivity is about 5 μl, while the response time is less than about 10 seconds. To improve the sensing range, cover layers may be applied to reserve liquid. Five variations of the biochemical sensor 100 with different cover layers were fabricated and tested, the sensing range and the response time for each sensor are recorded in Table 1. All values are to be construed as being about the expressed value.

TABLE 1

| Cover Layer | Sensing range | Response time |
|---|---|---|
| Single layer of cotton | 100 μl-300 μl | Less than 20 s |
| Single layer PVA hydrogel | 25 μl-150 μl | Less than 20 s |
| 5 layers of PVA hydrogel and filter | 100 μl-850 μl | More than 700 s |
| paper | | |
| 1 layer of PAA-PVA hydrogel | 25 μl-150 μl | 150 s-250 s |
| 2 layers of PAA-PVA and cotton | 100 μl-700 μl | Less than 100 s |

As shown in Table 1, the biochemical sensor 100 including a cover layer of 2 layers of PAA-PVA hydrogel and cotton demonstrated the best performance.

It should be understood that the sensitivity of the biochemical sensor 100 should be tailored to the application. When characterizing vaginal discharge, the biochemical sensor 100 should be able to detect acidity in biological liquids as small as about 0.5 ml to about 2 ml, which corresponds to the average vaginal discharge volume in one night (up to 12 hours).

It should be further understood that the sensitivity of the biochemical sensor 100 may depend on the gap distance 116 when the biochemical sensor 100 is configured to detect pH. The biochemical sensor 100 may only measure pH if a sufficiently large volume of liquid is deposited onto the flexible substrate 104, such that the biological liquid electrically connects the first and second electrodes. In some examples, the gap distance 116 is about 2 cm. In further examples, the gap distance 116 is about 1 cm. In further examples, the gap distance 116 is about 0.5 cm. In yet further examples, the gap distance 116 is about 0.1 cm. In further examples, the gap distance 116 is about 100 μm. Generally, the closer the first electrode 110 is to the second electrode 112, the greater the likelihood that the biochemical sensor 100 will be able to detect pH. Extrusion printing is advantageous because it can allow the electrodes 108 to be precisely placed with minimal gap distance.

In specific, non-limiting examples, the biochemical sensor 100 is configured to measure the volume of the biological liquid. In these examples, the first electrode 110 is a power electrode which is configured to receive a test signal from the microcontroller. The second electrode 112 is a sensing electrode which is configured to sense electrical current. If the biological liquid is deposited onto the flexible substrate 104 such that the first electrode 110 and the second electrode 112 are electrically connected by the biological liquid, the second electrode 112 transmits the feedback signal to the microcontroller. In some examples, the biological liquid is secreted by the user onto the flexible substrate 104. In examples where the biochemical sensor 100 is configured to measure the volume of the biological liquid, the electrodes 110, 112 typically comprise a conductive metal such as copper, gold, silver, or the like.

In some examples, the biochemical sensor 100 includes a plurality of the first electrodes 110. In some examples, the biochemical sensor 100 includes a plurality of the second electrodes 112. The microcontroller may be configured to determine the volume of the biological liquid based on the number of second electrodes 112 that transmit the feedback signal to the microcontroller, the gap distance between the first and second electrodes 110, 112, or a combination thereof. In examples where the volume is assessed according to the gap distance, the microcontroller may be configured to retrieve the gap distance from memory.

It should be understood that the sensitivity of the biochemical sensor 100 is correlated with the number of the electrodes 108 and the respective gap distances 116. Generally, if the first and second electrodes are positioned close together, the biochemical sensor 100 will be able to detect even small volumes of liquid. Furthermore, if the precision will be correlated with the number of electrodes 108 included in the biochemical sensor 100.

In specific, non-limiting examples, the biochemical sensor 100 is configured to measure the salt concentration of the biological liquid. In these examples, the first electrode 110 and the second electrode 112 may comprise interdigitated electrodes (IDEs) which are extrusion-printed onto the flexible substrate 104. The microcontroller is configured to apply a test signal to the first electrode 110 and receive a feedback signal from the second electrode 112. The microcontroller may include a conductivity meter for measuring the conductivity of the biological fluid between the first and second electrodes 110, 112, based on the feedback signal and the test signal. The microcontroller may be configured to determine the salt concentration of the biological liquid based on the conductivity.

In specific, non-limiting examples, the biochemical sensor 100 is configured to measure the antibody concentration of the biological liquid.

In some examples where the biochemical sensor 100 is configured to measure antibody concentrations, the electrodes 108 may comprise interdigitated electrodes (IDEs) which are extrusion-printed onto the flexible substrate 104. The first and second electrodes 110, 112 may be immobilized with anti-human IgA through any suitable technique including covalent immobilization via a self-assembled monolayer with high selectivity for IgA. The biochemical sensor 100 is configured to measure the interfacial charge transfer resistance (Rct) to determine the amount of IgA that is absorbed onto the immobilized anti-IgA. A wide range of antibody concentrations can be measured using IDEs, and in some examples, the biochemical sensor 100 can detect antibody concentrations between about 0.01 ng/ml and about 10000 ng/ml.

In other examples where the biochemical sensor 100 is configured to measure antibody concentrations, the electrodes 108 comprise interdigitated gold electrodes on a polyethylene naphthalate (PEN) film prepared with lithography. The sensitivity of the biochemical sensor 100 may be improved using the dense interdigitated electrodes and the gold surface may be modified by antigen molecules through several chemical methods.

In examples where the biochemical sensor 100 is configured to measure antibody concentrations, the electrodes 108 comprise a pair of carbon electrodes, nanostructured with a carbon-metal hybrid system that serves as the transducer. The carbon electrodes may be extrusion-printed or dual-screen printed onto the flexible substrate 104. The immuno-sensing strategy may involve the immobilization of CD-specific antigens on nanostructured electrode surfaces, such as gliadin or tTG, and human antibodies are detected electrochemically through the interaction between antigens and antibodies.

In specific, non-limiting examples, the biochemical sensor 100 is configured to measure the lactic acid concentration of the biological liquid. In these examples, the biochemical sensor 100 may comprise an enzymatic or non-enzymatic sensor. Preferably, the biochemical sensor 100 comprises a non-enzymatic sensor, which typically demonstrates higher efficiency, reliability and recyclability due to the absence of the biological component that is present in enzymatic sensors. This prevents biological degradation from occurring in these sensors. Though enzymatic sensors have higher sensitivity, a non-enzymatic sensor may be preferable due to its recyclability. Although, the lactic acid sensor may be also in the form of printed electronics or a flexible circuit or even integrated into the flexible substrate 104.

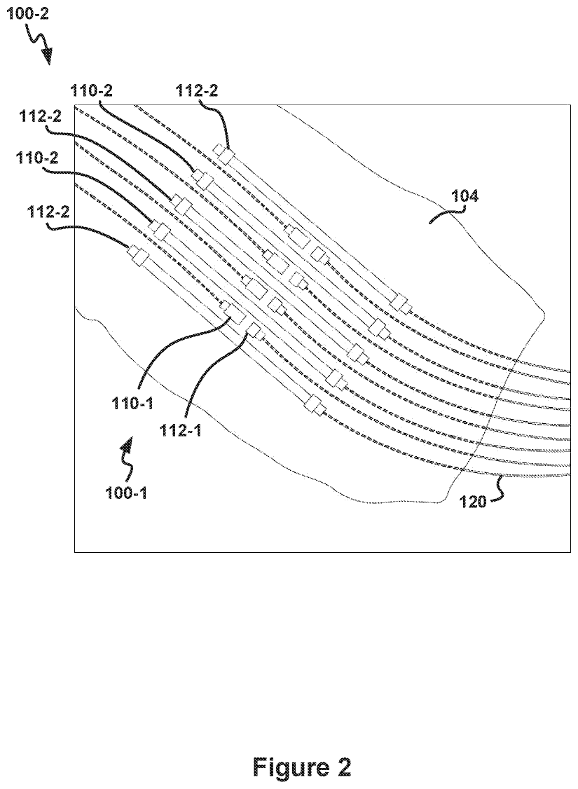

FIG. 2 is a schematic diagram showing variants of the biochemical sensor 100 from FIG. 1. In this embodiment, the flexible substrate 104 includes of a first sensor 100-1 and a second sensor 100-2, however the sensors are not particularly limited, and any suitable number of sensors may be printed onto the flexible substrate 104.

In this embodiment, the first sensor 100-1 comprises a first electrode 110-1 and a second electrode 112-1 proximal to the first electrode 110. In the embodiment shown in FIG. 2, the first sensor 100-1 is a pH sensor, however the first sensor 100-1 is not particularly limited.

The flexible substrate 104 may be printed with one, two, three, five, or any suitable number of the first sensor 100-1. The flexible substrate 104 shown in FIG. 2 is printed with four of the first sensor 100-1 which are spaced apart to improve the likelihood that a biological liquid deposited onto the flexible substrate 104 will contact at least one of the first sensors 100-1.

In the embodiment shown in FIG. 2, the second sensor 100-2 similarly comprises first electrodes 110-2 and second electrodes 112-2. In this embodiment, the second sensor 100-2 is a volume sensor, however the second sensor 100-2 is not particularly limited. The first electrodes 110-2 are power electrodes which apply a test signal comprising an electrical current to the biological liquid. The second electrodes 112-2 are sensing electrodes which receive feedback signals in the biological liquid and transmit the feedback signals to a microcontroller or similar device. In this example, the first electrodes 110-2 alternate with the second electrodes 112-2, however in other examples, only one of the electrodes 108 is a power electrode.

In the embodiment shown in FIG. 2, the first electrodes 110-2 and second electrodes 112-2 comprise elongate lines deposited onto the flexible substrate 104, however the electrodes 108 are not particularly limited to the arrangement shown in FIG. 2 and a number of configurations are contemplated. The electrodes 108 may comprise lines, dots, polygons, or combinations thereof. In examples where the electrodes 108 are lines, the electrodes 108 may be parallel. In further examples where the electrodes 108 are lines, the electrodes 108 may be straight lines, curved lines, zigzags, radial lines, abstract shapes, or a combination thereof.

The first sensor 100-1 and the second sensor 100 alternate in this embodiment, however other arrangements are contemplated.

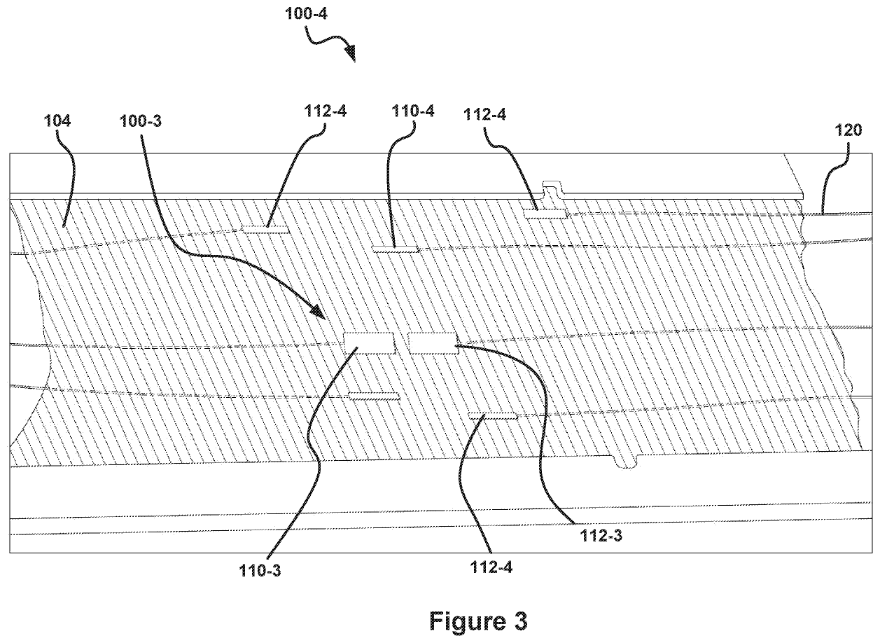

FIG. 3 is a schematic diagram of a third sensor 100-3 and a fourth sensor 100-4, which are variants of the biochemical sensor 100 of FIG. 1. In this example, the third sensor 100-3 is a pH sensor comprising a first electrode 110-3 and a second electrode 112-3. The third sensor 100-3 is positioned approximately in the center of the flexible substrate 104 in order to measure the pH of a liquid deposited near the center of the flexible substrate 104. In this example, the fourth sensor 100-4 is a volume sensor comprising one first electrode 110-4 and a plurality of second electrodes 112-4 which are spaced at varying distances from the first electrode 110-4. This arrangement may assist in measuring the volume of a biological liquid that is deposited near the center of the flexible substrate 104. The sensor 100-4 may receive feedback signals from one or more of the second electrodes 112-4 and determine the volume of the liquid according to the distance of the activated electrodes from the first electrode 110-4.

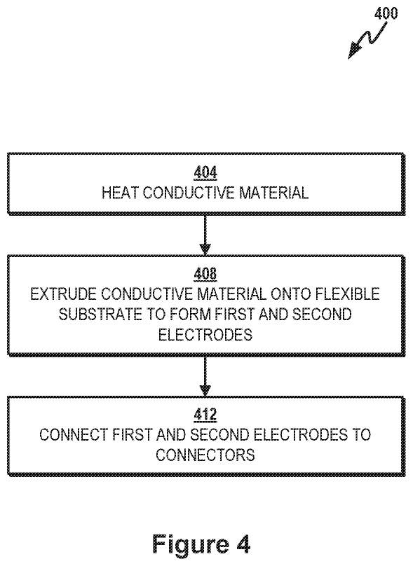

FIG. 4 is a block diagram of a method 400 for manufacturing the biochemical sensor 100. The method 400 may be performed by an extrusion printer which comprises a heating element for heating materials, a nozzle for extruding the heated material, and a controller for positioning the nozzle. The extrusion printer may further include a camera for aligning the nozzle. The extrusion printer may further include a build platform for supporting a substrate onto which the material is extruded. The build platform may be movable. The extrusion printer may include a vacuum chamber for stabilizing the substrate. The extrusion printer may further include a pressure sensor for detecting the pressure of the conductive material during extrusion. Responsive to the pressure sensor, the extrusion printer may control the pressure in order to control the resolution of the printing. A non-limiting example of an extrusion printer is the Voltera NOVA™, however other extrusion printers are known in the art.

The extrusion printer may be controlled by a computing device which is connected to the extrusion printer. The computing device may store instructions, that when executed, cause the extrusion printer to conduct method 400. In particular examples, the computing device includes memory storing three-dimensional design files which describe the layout of the biochemical sensor 100. The three-dimensional design files may specify the location the electrodes, the dimensions of the electrodes, the gap distances, the configuration of the IDEs, and the like.

Block 404 comprises heating the conductive material. Block 404 may be performed by the heating element which applies heat sufficient to evaporate solvents and solidify the conductive material. The heating temperature may vary according to the conductive material selected. For silver viscous inks, such as those described herein, the heating temperature is about 120° C. and the conductive material is heated for about 30 minutes, however block 404 is not particularly limited. The heating time and temperature may depend on the volume of ink and the design of the electrodes.

As part of block 404, the extrusion printer may receive material data pertaining to the conductive material. The material data may include the chemical composition and viscosity of the conductive material. Based on the material data, the extrusion printer may determine the printing parameters, which may include the heating temperature, the nozzle size, extrusion speed, nozzle height, and the like.

Block 408 comprises extruding the conductive material onto the flexible substrate 104 to print the first electrode 110 on the surface of the flexible substrate 104 and the second electrode 112 spaced from the first electrode 110 on the surface of the flexible substrate 104. Block 408 may be performed by the nozzle which deposits a layer of the conductive material to the flexible substrate 104. The nozzle size may be selected according to the desired resolution of the electrodes 108. In specific non-limiting examples, the nozzle has a diameter of about 250 μm. As a part of block 408, the controller may move the nozzle in order to position the first and second electrodes 110, 112 as desired. Block 408 is not particularly limited to two electrodes and may include forming any suitable number of electrodes on the flexible substrate.

Block 412 comprises connecting the first and second electrodes 110, 112 to respective connectors 120. In some examples, block 412 comprises connecting the electrode 108 to a plurality of the connectors 120. Block 412 may be performed manually or using robotics. In some examples, block 412 comprises embedding an end of the connector 120 into the electrode 108 before the conductive material cools and hardens. In other examples, block 412 comprises laying an end of the connector 120 on top of the electrode 108 and then repeating block 408 to form a second layer of the conductive material on top of the first layer such that the end of the connector 120 is sandwiched between the two layers. The opposite end of the connector 120 may be connected to a microcontroller or other suitable device for processing the feedback signals received at the second electrode 112.

Blocks 404 to 412 are repeated any suitable number of times to form any suitable number of sensors 100. The order of blocks shown in Figure is not particularly limited, and the method may be performed in any suitable order.

It will now be apparent to a person of skill in the art that the present specification affords certain advantages over the prior art. The screen-printed sensors of the prior art involve using a blade to spread ink through a mask containing the design and onto a substrate, a process that often results in significant ink wastage, which can be costly. Additionally, screen printing struggles with precision, particularly regarding the consistency of the material's thickness, the amount of material used, and placement of the electrodes. Moreover, the contact between the blade and the electrode makes contamination almost unavoidable. In contrast, extrusion printing is a non-contact method which is more suitable for health and medical applications in which sanitation is a priority. Extrusion printed sensors offer several other advantages over screen-printing. Sensors produced through extrusion printing are not only less wasteful but also exhibit consistent thickness and precise alignment. This method allows for narrower gaps between printed electrodes, leading to higher accuracy and efficiency in sensor performance.

FIG. 5 is a front elevation view of a wearable device 500 including the biochemical sensor 100 of FIG. 1. The wearable device 500 comprises a garment configured to be worn on the user's body. In some examples, the garment may comprise a plurality of fabric layers. In this example, the garment is underwear and the wearable device 500 comprises a front portion 502, a rear portion 504, a groin portion 504 and a waistband 508, however other configurations and types of garments are contemplated. In other examples, the garment comprises a shirt, pants, a bra, an undershirt, a headpiece, an armband, footwear, activewear, sleepwear, and the like.

The wearable device 500 further comprises a microcontroller 516 for receiving feedback signals from one or more input devices including the biochemical sensor 100. The input devices may further include a piezoelectric sensor 512, a temperature sensor 514, a viscoelasticity sensor, and the like.

The input devices may be disposed on the inner or outer surface of one of the fabric portions. In embodiments where the one or more of the fabric portions comprises a plurality of fabric layers, the input devices may be disposed between two layers. In certain other embodiments, the input devices are woven into the fabric of the wearable device 500. In the example shown in FIG. 5, the biochemical sensor 100 is disposed on the inner surface of the groin portion 506 while the piezoelectric sensor 512 is disposed along an edge of the leg opening 510, and the temperature sensor is disposed on the inner surface of the waistband 508.

To detect the properties of vaginal discharge, the biochemical sensor 100 may be located in the groin portion 506 of the wearable device 500 so that the biochemical sensor 100 is positioned to contact vaginal discharges. To detect the biochemical properties of sebum or sweat, the biochemical sensor 100 is preferably located in the front portion 502 or rear portion 504 of the wearable device 500 so that the biochemical sensor 100 is positioned to contact the user's skin, but the biochemical sensor 100 could instead be located in the waistband 508 or groin portion 506.

The piezoelectric sensor 512 may comprise a piezoelectric transducer, a piezoelectric belt, a piezoelectric nanofiber, or the like. The piezoelectric sensor 512 may detect the user's breath rate or pulse rate or a combination of both breath rate and pulse rate. In some examples, the wearable device 500 includes two piezoelectric sensors 512: a first piezoelectric sensor for measuring breath rate and a second piezoelectric sensor for measuring heart rate. The piezoelectric sensor 512 may be configured to measure low-frequency oscillations of the body indicative of breath rate.

In some examples, the piezoelectric sensor 512 is formed by extrusion-printing a piezoelectric material onto the flexible substrate 104. The piezoelectrical material may comprise polyvinylidene fluoride (PVDF), lead zirconate titanate (PZT), lead zirconate titanate (KNN), barium titanate (BTO), or a combination thereof. After extrusion printing, the piezoelectric material may be further treated with post-processing treatments such as Selective Laser Sintering (SLS). SLS may fuse the particles of the piezoelectric material, enhancing the material's structural integrity and activating piezoresistive characteristics. This post-processing step may enhance the functionality and reliability of the piezoelectric sensor 512.

The piezoelectric sensor 512 may be located along the edge of the leg opening 510, as shown in FIG. 5. The advantage of this position is that the piezoelectric sensor 512 is proximal to the user's femoral artery when the device 500 is worn, and thus a more accurate heart or pulse rate may be measure, however the piezoelectric sensor 512 is not particularly limited.

In examples where the piezoelectric sensor 512 comprises a piezoelectric belt, the piezoelectric sensor 512 may be located in the waistband 508 of the wearable device. The piezoelectric belt may be configured to detect electrocardiogram (ECG) signals indicative of pulse rate, respiratory signals, or a combination of both.

The temperature sensor 514 generally comprises a thermal sensor for measuring the skin temperature of the user. The temperature sensor 514 generally comprises a temperature-sensitive material connected to a pair of electrodes. The pair of electrodes may be extrusion printed (as described above with respect to electrodes 108) or the pair of electrodes may comprise conductive yarn sewn into the fabric. In examples where the wearable device 500 includes a temperature sensor 514, the microcontroller 516 is configured to measure an electrical property of the temperature-sensitive material across the two electrodes, and the microcontroller 516 is configured to determine the skin temperature of the user based on the electrical property.

The temperature-sensitive material may be applied to the flexible substrate 104 by extrusion printing, screen printing, inkjet printing, drop casting, or dip coating.

In some examples, the temperature-sensitive material comprises a multi-walled carbon nanotube (MWCNT). In these examples, the electrical property is conductivity and the microcontroller 516 includes a conductivity meter for measuring the conductivity of the MWCNT between the two electrodes MWCNT sensors are suitable for continuous skin temperature monitoring because they demonstrate a good response rate, are easily fabricated, and are waterproof.

In other examples, the temperature sensor 514 comprises a negative temperature coefficient (NTC) sensor, and the temperature-sensitive material comprises a semiconductor material which decreases in resistance as temperature increases, and vice versa. The semiconductor material may include a metal oxide, a polymer, silicon, or compositions thereof. In specific non-limiting examples, the semiconductor material comprises PEDOT: PSS. The temperature-sensitive material may further include at least one of poly(3,4-ethylenedioxythiophene) (GOPS), 4-Dodecylbenzenesulfonic acid (DBSA), deionized water (DI water), and ethylene glycol. In examples where the temperature sensor 514 is a NTC sensor, the electrical property is resistance, and the microcontroller 516 include an ohmmeter for measuring the resistance of the temperature-sensitive material between the two electrodes.

According to one embodiment of the NTC sensor, the temperature-sensitive material comprises PEDOT:PSS and graphene oxide (GO). The temperature sensor 514 was manufactured by sonicating the GO in deionized (DI) water and mixing the GO with PEDOT: PSS to obtain a mixture having a ratio of 1:1 (PEDOT:PSS and GO). The mixture is stirred at about 900 RPM for about 1 hour. In this example, the temperature sensor 514 is thin and flexible. The temperature-sensitive material is printed onto the flexible substrate 104 using extrusion printing. Two electrodes comprising silver ink were extrusion printed adjacent to the temperature-sensitive material. The sensor 514 was shown to detect temperatures ranging between room temperature (22° C.) and 50° C., which is a suitable range for measuring skin temperature.

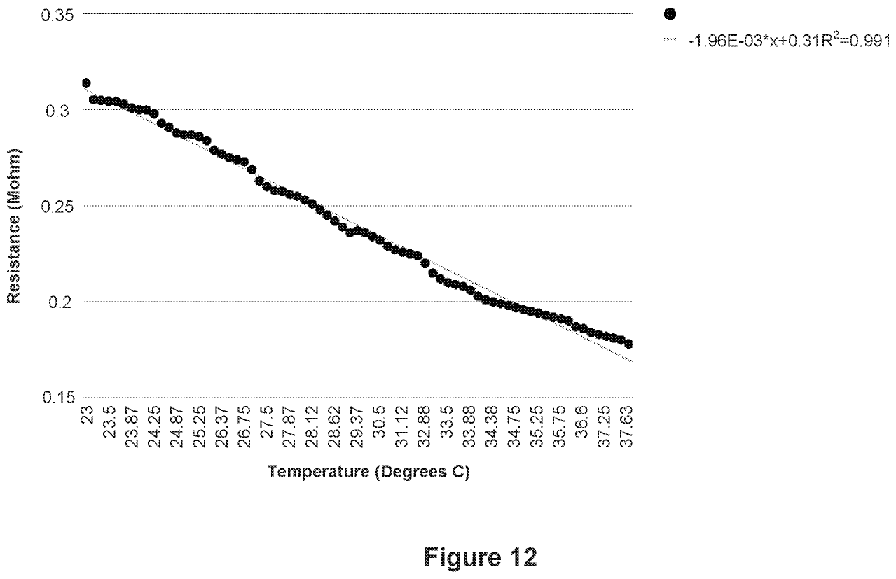

FIG. 12 is a graph showing the resistance of the temperature sensor 514 according to one embodiment. In this example, the temperature sensor 514 is an NTC sensor and the temperature-sensitive material comprises PEDOT: PSS deposited on a fabric substrate. Conductive yarn sewn onto the fabric serves as the two electrodes which are connected to an ohmmeter for measuring the resistance. The temperature sensor 514 was placed on a hot plate to vary the temperature while measuring the resistance. The temperature of the temperature sensor 514 was detected using a reference temperature sensor (Adafruit PCT2075™ Temperature Sensor) which was positioned adjacent to the temperature sensor 514. As shown in FIG. 12, the relationship between the resistance of the temperature-sensitive material and temperature is linear. By fitting a linear curve to the data, a formula can be derived that allows for the determination of temperature based on the resistance. The accuracy of the temperature sensor 514 was determined to be 99% and the sensitivity was calculated to be 107.7 kΩ/° C.

In a further non-limiting example, the temperature sensor 514 comprises a positive temperature coefficient (PTC) sensor. In these examples, the temperature-sensitive material comprises a material that increases in resistance as temperature increases, and vice versa. In specific non-limiting examples, the temperature-sensitive material is comprised of several polymers and conductive filler and is shown to be both thin and flexible. This sensor has been shown to have temperature measurement ranges from 25° C. to 45° C. which is appropriate for measuring skin temperature. PTC sensors can be lightweight and inexpensive to fabricate. The PTC sensor changes in its resistivity as temperature increases and these changes are used to measure the temperature. In examples where the temperature sensor 514 is a PTC sensor, the electrical property is resistance, and the microcontroller 516 include an ohmmeter for measuring the resistance of the temperature-sensitive material between the two electrodes.

While the temperature sensor 514 has been described above with respect to sensors printed on the flexible substrate, it should be understood that alternatives are contemplated. For example, the temperature sensor may be sewn or glued to the garment. In one non-limiting example, the temperature sensor 514 comprises the Adafruit PCT2075™ Temperature Sensor (Adafruit: New York, United States).

To detect the skin temperature, the temperature sensor 512 is preferably located on an inner surface of the wearable device 500 so as to contact the skin of the user. In some examples, the temperature sensor 512 is located on an inner surface of the waistband 508. In other examples, the temperature sensor 512 is located along the edge of the leg opening 510 so that the temperature sensor 512 is proximal to the femoral artery.

The microcontroller 516 is configured to determine a physiological parameter based on the feedback signals received from the one or more input devices 100, 512, 514. The physiological parameter may include acidity (pH) of a biological liquid, skin temperature, breath rate, pulse rate, salt content of biological liquid, lactic acid content of biological liquid, antibody content of biological liquid, viscoelasticity of biological liquid, volume of biological liquid, and the like. The input device generates the feedback signal indicative of the physiological parameter and transmits the feedback signal to the microcontroller 516 via the connector 120. In examples where the wearable device 500 includes more than one input device, the wearable device 500 can generate feedback data pertaining to more than one physiological parameter. Each of the input devices may detect a different physiological parameter and may be positioned according to the respective physiological parameter.

The connector 120 connects the input device to the microcontroller 516. The connector 120 may be disposed between two layers of fabric, disposed on the surface of a layer of fabric, sewn into the fabric, or the connector may be woven into the fabric of the wearable device 500.

The microcontroller 516 is preferably located in the waistband 508 of the wearable device 500, but the microcontroller 516 is not particularly limited. Microcontroller 516 receives the feedback signal from the input device and generates feedback data based on the feedback signal. The microcontroller 516 may include a Microcontroller Unit (MCU) such as the Arduino™ UNO (Arduino: New York, United States) or the Arduino™ Nano 33 BLE (Arduino: New York, United States), however the microcontroller 516 is not particularly limited.

In some examples, the wearable device 500 does not include a microcontroller 516 and instead includes a wireless transmitter for transmitting the feedback signal wirelessly. Suitable examples of a wireless transmitter include a Wi-Fi module, a Bluetooth™ module, radiofrequency identification (RFID) tag, and the like.

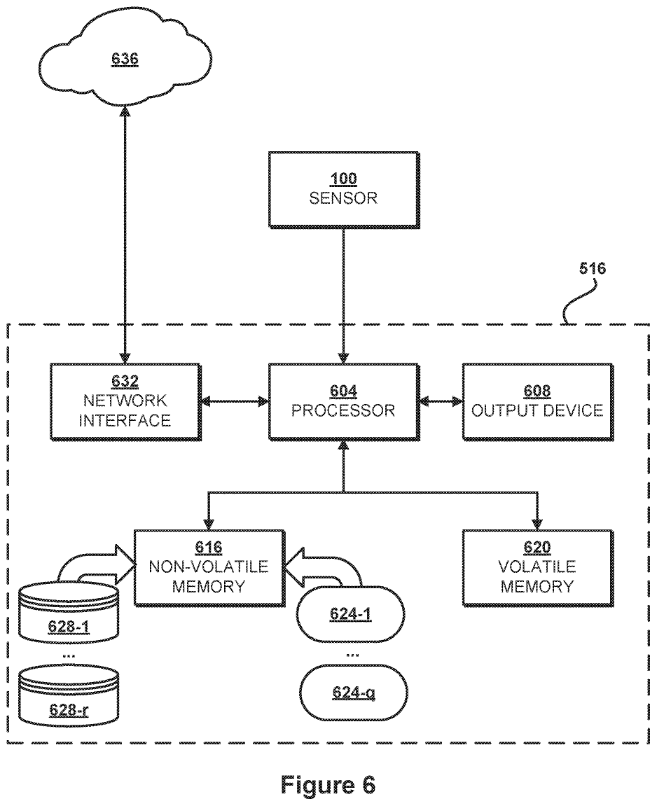

FIG. 6 in a schematic diagram of the wearable device 500 showing the microcontroller 516 is greater detail. The microcontroller 516 may comprise a processor 604 for receiving the feedback signal from the input device and processing said feedback signal to generate an output.

The processor 604 may be implemented as a plurality of processors or one or more multi-core processors. The processor 604 may be configured to execute different programing instructions responsive to the input received via the biochemical sensors 100 and to control one or more output devices 608 to generate output on those devices.

To fulfill its programming functions, processor 604 is configured to communicate with one or more memory units, including non-volatile memory 616 and volatile memory 620. Non-volatile memory 616 can be based on any persistent memory technology, such as an Erasable Electronic Programmable Read Only Memory (“EEPROM”), flash memory, solid-state hard disk (SSD), other type of hard-disk, or combinations of them. The non-volatile memory 616 may also be described as a non-transitory computer readable media. Also, more than one type of non-volatile memory 616 may be provided.

The volatile memory 620 is based on any random-access memory (RAM) technology. For example, the volatile memory 620 can be based on a Double Data Rate (DDR) Synchronous Dynamic Random-Access Memory (SDRAM). Other types of volatile memory 620 are contemplated.

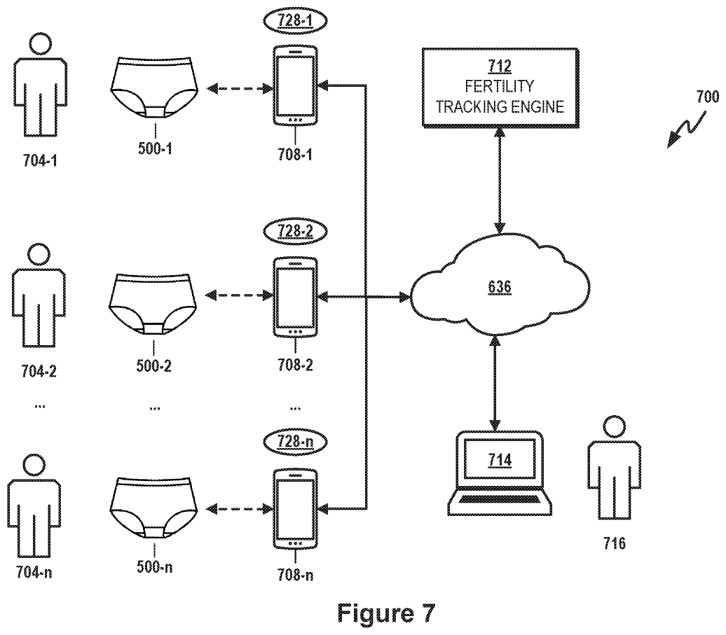

The processor 604 also connects to network 536 via a network interface 632. The network interface 632 can also be used to connect another computing device that has an output device, thereby obviating the need for output device 608 altogether. Suitable examples of network interfaces may include a Wi-Fi module, a Bluetooth™ module, RFID tag, the like, or a combination thereof. Suitable computing devices will be described in greater detail with respect to FIG. 7.

Programming instructions in the form of applications 624 are typically maintained, persistently, in non-volatile memory 616 and used by the processor 608 which reads from and writes to volatile memory 620 during the execution of applications 624. Various methods discussed herein can be coded as one or more applications 624 (generically referred to herein as “application 524” or collectively as “applications 624”).

One or more tables or databases 628 are maintained in non-volatile memory 616 for use by the applications 624.

The microcontroller 516 further comprises a power source (not shown) for powering the biochemical sensor 100 and the microcontroller 516. The power source may include a battery, a power port, a self-charging power pack, a power source that converts body energy into electricity, or a combination thereof. In examples where the source is a battery, the battery may be rechargeable or non-rechargeable battery. The battery may be removable or permanent. In embodiments where the microcontroller includes a power port for receiving power from an external source, the power source may further include a battery, and the power port may be configured to charge said battery.

In certain non-limiting examples, the power source comprises one or more lithium-ion batteries to power the sensors, resistors, switches and MCU. This power source is connected to a breadboard and transferred to the MCU, and other components attached. The microcontroller 516 may be powered by the serial USB port from the computer station. The accompanying batteries might be stored in a 3D-printed box and located on the wrist of the user ensuring comfort and safety while using the device.

In other non-limiting examples, the power source comprises the series DMW-BLF19. The 7.2V, 1860 mAh battery potentially works for up to 24 hours if the operating voltage of the microcontroller 516 is between 7 to 14 V. Other power sources such as fully self-charging power packs (FSPP) or power sources that work use body heat to store energy and use that can be other power sources used in this system.

In further non-limiting examples, the power source comprises a Molex™ electronic battery (Mouser Electronics: Kitchener, Canada) which is a very light and thin battery. This thin-film battery may power the microcontroller and the input devices. The Molex™ battery has a shelf life of about two years and can operate in a humidity of about 20% to about 90% and in a temperature range of about −35° C. to about 50° C. The Molex™ battery is a 3V battery with an initial internal resistance of about 90 ohms and a peak current (maximum) of about 8 to about 10 mA. The Molex™ battery is bendable and small, having a minimum bending radius of about 35.00 mm, a thickness of about 0.70 mm, and a width of about 36.00 mm.

FIG. 7 is a schematic diagram of a system 700 for managing feedback data representing physiological parameters. System 700 comprises a plurality of wearable devices 500, each wearable device 500 associated with a respective user 704. In the system 700, the wearable devices 500 transmit feedback data to respective computing devices 708. The network 636 interconnects the computing devices 708 with a fertility tracking engine 712 and an administrator terminal 714. As will be discussed further below, the fertility tracking engine 712 performs a number of processing functions for the system 700. For exemplary purposes, the embodiment shown in FIG. 7, will be described with respect to fertility, however the system is not particularly limited to fertility, and other health statuses are contemplated.

The computing devices 708 can be any type of human-machine interface for interacting with the wearable device 500. For example, computing devices 708 may include smartphones, personal computers, tablet computers, smartwatches, smart home systems, and any other device that can be used to receive and send content that complement the input and output hardware devices associated with a given computing device 608. The computing devices 708 can be operated by different users 704 that are associated with a respective identifier object 728 that uniquely identifies a given user 704 accessing a given computing device 708 in the system 700.

A person of skill in the art is to recognize that the form of an identifier object 728 is not particularly limited, and in a simple example embodiment, can be simply an alpha-numerical sequence that is entirely unique in relation to other identifier objects in the system 700. The identifier objects 728 can also be more complex as they may be combinations of account credentials (e.g. username, password, Two-factor authentication token, etc.) that uniquely identify a given user 704. The identifier objects 728 themselves may also be indexes that point to other identifier objects, such as one or more user accounts. The salient point is that they are uniquely identifiable within the system 700 in association with what they represent.

In specific embodiments, computing devices 708 may further generate user-reported data in response to inputs received at an input device, said inputs representing one or more health parameters. User-reported data may include dietary sugar, current age, ethnicity, age of first menstruation, method of birth control, reproductive and hormonal disorders, mood, pain, sleep quality, sleep duration, sex drive, sexual activity, menstrual bleeding, spotting, vaginal discharge, skin condition, cravings, digestion, weight, hair condition, the like, and a combination thereof.

The fertility tracking engine 712 can be based on any present or future electronic servers or computing architectures that, amongst other things, manage feedback data representing physiological parameters, and in particular examples, physiological parameters of reproductive cycles.

The administrator terminal 714 can also be any type of human-machine interface of the same types as computing devices 708. The administrator terminal 714 is operated by an administrator 716.

In a present example embodiment, the users 704 may wish to identify their present reproductive phase or predict the onset of a future reproductive phase. A user 704 may thus wear the wearable device 500 which collects feedback signals and generates feedback data representing at least one physiological parameter. The feedback data is transmitted via the network 736 to the fertility tracking engine 712 which processes the feedback data using an algorithm to generate reproductive data about the user. The reproductive data is transmitted via the network 736 and output at the computing device 608. The administrator 716 operates the administrator terminal 714 to input the algorithm, and optionally, training data to train the algorithm.

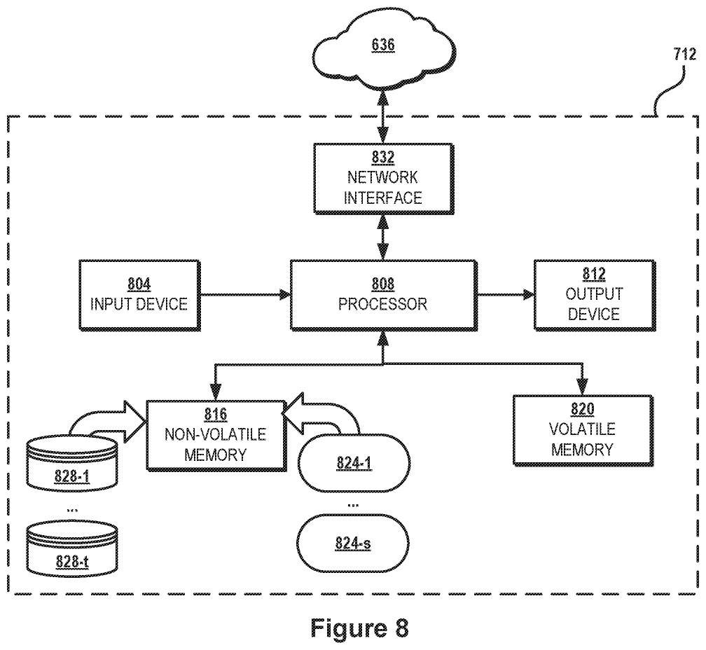

Having described an overview of the system 700, it is useful to comment on the hardware infrastructure of the system 700. FIG. 8 shows a schematic diagram of a non-limiting example of internal components of fertility tracking engine 712.

In this example, the fertility tracking engine 712 includes at least one input device 804. Input from the input device 804 is received at a processor 808 which in turn controls an output device 812. The input device 804 can be a traditional keyboard and/or mouse to provide physical input. Likewise, the output device 812 can be a display. In variants, additional and/or other input devices 804 or output devices 812 are contemplated or may be omitted altogether as the context requires.

The processor 808 may be implemented as a plurality of processors or one or more multi-core processors. The processor 808 may be configured to execute different programming instructions responsive to the input received at the one or more input devices 804 and to control one or more output devices 812 to generate output on those devices.

To fulfill its programming functions, the processor 808 is configured to communicate with one or more memory units, including non-volatile memory 816 and volatile memory 820. The non-volatile memory 816 can be based on any persistent memory technology, such as an Erasable Electronic Programmable Read Only Memory (“EEPROM”), flash memory, solid-state hard disk (SSD), other type of hard-disk, or combinations of them. The non-volatile memory 816 may also be described as a non-transitory computer readable media. Also, more than one type of non-volatile memory 816 may be provided.

The volatile memory 820 is based on any random-access memory (RAM) technology. For example, the volatile memory 820 can be based on a Double Data Rate (DDR) Synchronous Dynamic Random-Access Memory (SDRAM). Other types of volatile memory 820 are contemplated.

The processor 808 also connects to network 636 via a network interface 832. The network interface 832 can also be used to connect the processor 808 to another computing device that has an input and output device, thereby obviating the need for the input device 804 and/or the output device 812 altogether.

Programming instructions in the form of applications 824 are typically maintained, persistently, in non-volatile memory 816 and used by the processor 808 which reads from and writes to volatile memory 820 during the execution of applications 824. Various methods discussed herein can be coded as one or more applications 824. One or more tables or databases 828 are maintained in the non-volatile memory 816 for use by the applications 824.

The infrastructure of the fertility tracking engine 712, or a variant thereon, can be used to implement any of the computing nodes in the system 700, including computing devices 708. Furthermore, the fertility tracking engine 712 and computing devices 708 may be implemented as virtual machines and/or with mirror images to provide load balancing. Functions of the fertility tracking engine 712 may also be distributed amongst different computing devices 708, thereby obviating the need for a central fertility tracking engine. By the same token, a plurality of fertility tracking engines 712 may be provided.

Furthermore, a person of skill in the art will recognize that the core elements of the processor 808, the input device 804, the output device 812, the non-volatile memory 816, the volatile memory 820 and the network interface 832, as described in relation to the server environment of the fertility tracking engine 712, have analogues in the different form factors of computing device machines such as those that can be used to implement the computing devices 708 and the administrator terminal 714.

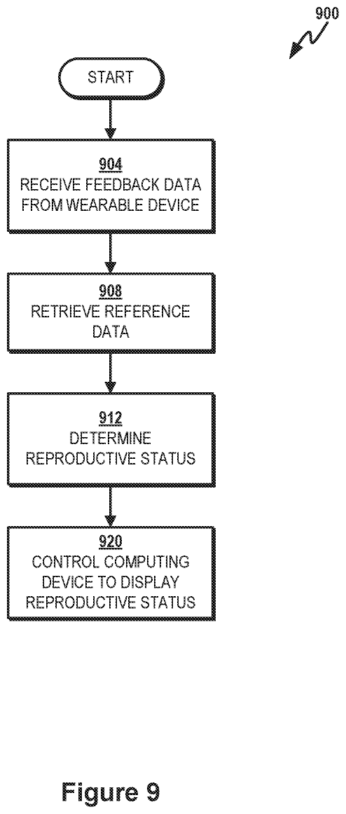

FIG. 9 shows a flowchart depicting a method for generating reproductive data indicated generally at 900. The method 900 can be implemented on the system 700. Persons skilled in the art may choose to implement the method 800 on the system 700 or variants thereon, or with certain blocks omitted, performed in parallel or in a different order than shown. The method 900 can thus also be varied. However, for purposes of explanation, the method 900 will be described in relation to its performance on the system 700 with a specific focus on treating the method 900 as, for example, application 824-1 maintained within fertility tracking engine 712 and its interactions with the other nodes in the system 700.

Block 904 comprises receiving feedback data. The feedback data comprises at least a physiological parameter corresponding to the type of sensor from which the corresponding feedback signal was received, as previously described with respect to the input devices of FIG. 5, and a value for said physiological parameter, but the feedback data is not particularly limited. The feedback data may further include the date or dates on which the feedback signal was received at the wearable device 500. The feedback system may further include user reported data input at the computing device 608. In the system 700, the fertility tracking engine 712 receives the feedback data from computing device 708.

At block 908, the fertility tracking engine 712 retrieves reference data from memory 816. The reference data comprises both temporal data and physiological data. Temporal data may comprise a phase or a day within a reproductive cycle. Phases of the reproductive cycle include luteal phase, follicular phase, ovulation, fertile phase, proliferative phase, secretory phase, period, pregnancy, and the like. A day may be indicated as “Day 1 of 31” or the like. Physiological data may comprise a physiological indicator corresponding with the respective temporal data. In a specific example, the physiological data may comprise average body temperatures corresponding to days of the reproductive cycle.

The reference data may be input by the administrator 716 at the administrative terminal 714 and transmitted to the fertility tracking engine 712 where it is stored in the memory 816. A person of skill in the art will understand that the reference data may represent the average human reproductive cycle. In specific embodiments, the reference data may be selected according to the user-reported data and the reference data may represent the average reproductive cycle for a user of a particular age, weight, ethnicity, hormonal disorder, or the like.

At block 912, the fertility tracking engine 712 determines the reproductive status of the user based on a comparison between the feedback data and the reference data. In general, the reproductive status represents a particular day or phase in the user's reproductive cycle. The fertility tracking engine 712 determines the reproductive status by applying the application 824 that compares the feedback data to the reference data. The comparison may comprise comparing the physiological parameters present in the feedback data with the physiological data in the reference data and selecting the reference data that most closely resembles the feedback data. The application 824 may then determine the user's reproductive status based on the selected reference data.

In addition to determining a reproductive status, block 912 may comprise determining a disease condition such as endometriosis, uterine fibroids, gynecologic cancer, polycystic ovary syndrome, congenital adrenal hyperplasia, sexually transmitted diseases, and the like.

In some examples, the applications 824 may include machine learning and/or deep-learning-based algorithms and/or neural networks, and the like, which are trained to improve the accuracy of the reproductive status determined at block 912. Furthermore, in these examples, applications 824 may be operated by the fertility tracking engine 712 in a training mode to train the machine learning and/or deep-learning based algorithms and/or neutral networks of applications 824 in accordance with the teachings herein.

The one or more machine-learning algorithms and/or deep learning algorithms and/or neural networks of the applications 824 may include, but are not limited to: a generalized linear regression algorithm; a random forest algorithm; a support vector machine algorithm; a gradient boosting regression algorithm; a decision tree algorithm; a generalized additive model; neural network algorithms; deep learning algorithms; evolutionary programming algorithms; Bayesian inference algorithms; reinforcement learning algorithms, and the like. However, generalized linear regression algorithms, random forest algorithms, support vector machine algorithms, gradient boosting regression algorithms, decision tree algorithms, generalized additive models, and the like may be preferred over neural network algorithms, deep learning algorithms, evolutionary programming algorithms, and the like. However, generalized linear regression algorithms, random forest algorithms, support vector machine algorithms, gradient boosting regression algorithms, decision tree algorithms, generalized additive models, and the like may be preferred over neural network algorithms, deep learning algorithms, evolutionary programming algorithms, and the like. To be clear, any suitable machine-learning algorithm and/or deep learning algorithm and/or neural network is within the scope of present examples.

Such machine learning and/or deep-learning based algorithms can, for example, track feedback data and user-generated data associated with the users of each the wearable device 500. With sufficient training, the algorithm can begin to determine patterns in the presentation of physiological parameters in a particular user or a demographic of users.

As part of block 912, and in response to determining the reproductive status, the fertility tracking engine 712 may forecast the reproductive cycle of the user. Forecasting the reproductive cycle consists of generating a prediction for one or more future reproductive statuses. The forecast may comprise an estimated date for beginning a reproductive phase. In some examples, the forecast may comprise a timeline of future dates and the reproductive status predicted for each of the future dates. The forecast may be generated by the application 824 based on the reproductive status determined at block 912 and the reference data.

The forecast may include a predicted volume of menstrual flow, prediction of when a sanitary product for collecting menstrual flow will need to be replaced, predicted onset of reproductive symptoms, the like, or a combination thereof.

In a specific, non-limiting example, the application 824 compares the user's physiological parameters to the reference data and determines at block 912 that the user is currently on Day 26 of the reproductive cycle. Then, the application 824 compares the reproductive status to the reference data and predicts that the user will begin menstruation in two days and begin ovulation in 12 days.

Block 920 comprises controlling the computing device 708 to display the reproductive status. In the system 700, block 920 is performed by the fertility tracking engine 712 which controls the computing device 708 to display the reproductive status at the output device 608. The reproductive status may be output as a graphic, text, audio, or the like. In some examples, the reproductive status may be output as an alert.

The method 900 may be repeated on a continual or periodic basis. In some examples, the method 900 is performed in response to receiving feedback data from the wearable device 500. In other examples, method 900 is performed periodically.

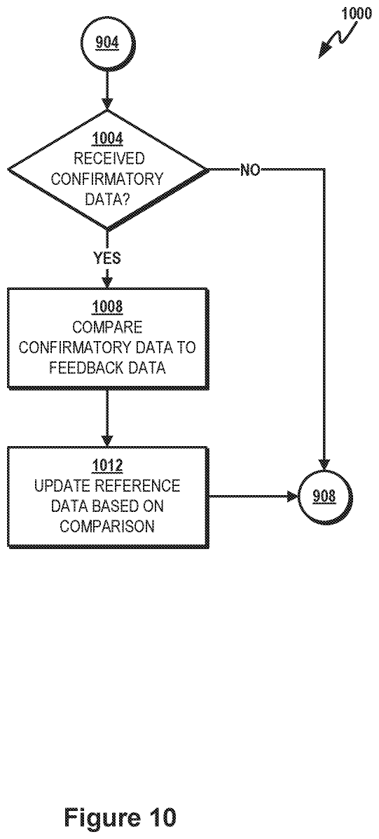

In examples where the application 824 comprises a machine learning algorithm, the application 824 is trained with confirmatory data. FIG. 10 is a block diagram showing an exemplary method 1000 of training the machine learning algorithm.

In the example shown in FIG. 10, the method 1000 is performed after receiving feedback data at block 904, however method 1000 is not particularly limited. Method 1000 may be performed any suitable time. In some examples, method 1000 performed after determining a reproductive status at block 912. In further examples, method 1000 is performed in response to receiving confirmatory data.

At block 1004, the fertility tracking engine 712 receives confirmatory data. In system 700, block 1004 is performed by fertility tracking engine 712 which receives confirmatory data input at computing device or at the administrative terminal 714.

Confirmatory data may comprise the result of a biochemical test such as a urine or blood test, a physical examination by a medical professional, an ultrasound, the like, or a combination thereof. In some examples, the confirmatory data may comprise user-reported data. In general, the confirmatory data is selected to verify the reproductive status of the user, however the confirmatory data is not particularly limited. The confirmatory data may further include information about a disease state. A person of skill in the art will understand that the confirmatory data is preferably a reliable and accurate method of assessing the reproductive status of the user such a hormone test for pregnancy or ovulation, or the discharge of blood.

At block 1008, the fertility tracking engine 712 compares the confirmatory data with the feedback data received at block 904. The comparison may further comprise comparing the confirmatory data with user-generated data.

At block 1012, the fertility tracking engine 712 updates the reference data based on the comparison from block 1008. The updated reference data may comprise the confirmatory data and associated feedback data. The updated reference data is stored in the memory 816 at the fertility tracking engine 712. In some examples, the updated reference data may be stored in association with the user identifier associated with the confirmatory data. In some examples, the updated reference data may be stored in association with the user's demographic data.

A person of skill in the art will understand that the input of confirmatory data can improve the accuracy of the reproductive status determined at block 912. As the fertility tracking engine 712 collects confirmatory data, the application 824 may generate reproductive forecasts that are tailored to a particular user or demographic. The application 824 may learn to identify variations in a user's reproductive cycle and generate forecasts according to those variations. In a specific, non-limiting example, the application 824 may learn to predict when a user's menstrual flow will be heavier than usual or when the user's fertile window is going to be shorter than usual.

In view of the above, it will now be apparent that variant, combinations, and subsets of the foregoing embodiments are contemplated. For example, while the wearable device 500 and the system 700 were discussed above in relation to determining the reproductive status of a user, other health statuses are contemplated. In some examples, the device 500 and the system 700 may be used for disease detection, fitness tracking, skin health, hydration monitoring, nutrition planning, stress detection, and the like. In these examples, the fertility tracking engine 712 may be a health tracking engine configured to determine the health status of the user.

It will now be apparent to a person of skill in the art that the present specification affords certain advantages over the prior art. Firstly, the system 700 provides a non-invasive method of continuously monitoring physiological parameters to generate a prediction for the user's current or future reproductive status. Secondly, the wearable device 500 can be easy washed and re-used, which reduces the waste associated with current methods of fertility monitoring. Lastly, by monitoring multiple physiological parameters on an ongoing basis, the wearable device 500 and the system 700 can produce more accurate predictions for a user's reproductive cycle.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

1. A biochemical sensor comprising:
a flexible substrate; and
a plurality of electrodes comprising a conductive material extrusion-printed onto the flexible substrate, the plurality of electrodes including a first electrode and a second electrode spaced from the first electrode; and
a microcontroller connected to the plurality of electrodes, the microcontroller configured to:
receive a feedback signal from the second electrode responsive to a biochemical property of a biological liquid contacting at least the first electrode and the second electrode; and
determine the biochemical property of the biological liquid based on the feedback signal.

2. The biochemical sensor of claim 1 wherein the conductive material comprises at least one of silver, gold, copper, carbon nanotubules (CNT), reduced graphene oxide (rGO), polyaniline (PANI), poly(3,4-ethylenedioxythiophene), and polystyrene sulfonate (PEDOT: PSS).

3. The biochemical sensor of claim 1 wherein the flexible substrate comprises a fabric blend including polyester and cotton.

4. The biochemical sensor of claim 1, wherein the biochemical property is the acidity of the biological liquid, and the microcontroller further comprises a potentiostat configured to measure the potential across the first electrode and the second electrode to determine the acidity of the biological liquid.

5. The biochemical sensor of claim 1 wherein the biochemical property is the volume of the biological liquid, and wherein the microcontroller is configured to:
apply a test signal to the first electrode;
receive the feedback signal from the second electrode, the feedback signal responsive to the test signal;
retrieve from memory a gap distance between the first and second electrode; and
determine the volume of the biological liquid based on a gap distance between the first electrode and the second electrode.

6. The biochemical sensor of claim 5 further comprising a plurality of the second electrodes, wherein the microcontroller is further configured to determine the volume of the biological liquid based on the number of second electrodes transmitting the feedback signal.

7. The biochemical sensor of claim 1 wherein the biochemical property is the salt concentration of the biological liquid, wherein the microcontroller is further configured to
apply a test signal to the first electrode;
receive the feedback signal from the second electrode, the feedback signal responsive to the test signal; and
measure the conductivity of the biological liquid based on the test signal and the response signal using a conductivity meter; and
determine the salt concentration of the biological liquid based on the conductivity.

8. A wearable device comprising:
a garment configured to be worn by a user, the garment comprising a flexible substrate;
a biochemical sensor comprising a plurality of electrodes, the plurality of electrodes comprising a conductive material extrusion-printed onto the flexible substrate, the plurality of electrodes including a first electrode and a second electrode spaced from the first electrode;
a microcontroller connected to the plurality of electrodes, the microcontroller configured to:
receive a feedback signal from the second electrode responsive to a biochemical property of a biological liquid contacting at least the first electrode and the second electrode; and
determine the biochemical property of the biological liquid based on the feedback signal.

9. The wearable device of claim 8 wherein the conductive material comprises at least one of silver, gold, copper, carbon nanotubules (CNT), reduced graphene oxide (rGO), polyaniline (PANI), and poly(3,4-ethylenedioxythiophene) polystyrene sulfonate (PEDOT: PSS).

10. The wearable device of claim 8 wherein the flexible substrate comprises a fabric blend including polyester and cotton.

11. The wearable device of claim 8 wherein the biochemical property is the acidity of the biological liquid, and the microcontroller further comprises a potentiostat configured to measure the potential across the first electrode and the second electrode to determine the acidity of the biological liquid.

12. The wearable device of claim 8 wherein the biochemical property is the volume of the biological liquid, and wherein the microcontroller is configured to:
apply a test signal to the first electrode;
receive the feedback signal from the second electrode, the feedback signal responsive to the test signal; and
determine the volume of the biological liquid based on a gap distance between the first electrode and the second electrode.

13. The wearable device of claim 12 further comprising a plurality of the second electrodes, wherein the microcontroller is further configured to determine the volume of the biological liquid based on the number of second electrodes transmitting the feedback signal.

14. The wearable device of claim 8 wherein the biochemical property is the salt concentration of the biological liquid, and the microcontroller further comprises a conductivity meter configured to:
measure the conductivity of the biological liquid between the first electrode and the second electrode; and
determine the salt concentration of the biological liquid based on the conductivity.

15. The wearable device of claim 8 further comprising a wireless transmitter connected to the microcontroller, the wireless transmitter configured to transmit the feedback signal via a network.

16. The wearable device of claim 8 further comprising a temperature sensor positioned to contact the user's skin, the temperature sensor comprising:
a pair of electrodes connected to the microcontroller; and
a temperature-sensitive compound extrusion-printed onto the flexible substrate and connected to the pair of electrodes;
wherein the microcontroller is configured to measure an electrical property of the temperature-sensitive material and determine the skin temperature of the user based on the electrical property.

17. The wearable device of claim 16 wherein the temperature-sensitive compound comprises poly(3,4-ethylenedioxythiophene) polystyrene sulfonate (PEDOT:PSS), Graphene Oxide (GO), and wherein the pair of electrodes comprise silver.

18. The wearable device of claim 8 further comprising a piezoelectric sensor connected to the microcontroller and configured to transmit piezoelectric signals to the microcontroller responsive to the user's heart rate or breath rate.

19.-32. (canceled)
